(19) United States
(12) Patent Application Publication
(10) Pub. No.: US 2023/0407220 A1
(43) Pub. Date: Dec. 21, 2023

(54) NOVEL TISSUE CULTURE SYSTEMS AND REDUCED GRAVITY CULTURE METHOD FOR THE PRODUCTION OF VASCULARIZED TISSUE

(71) Applicant: THE REGENTS OF THE UNIVERSITY OF CALIFORNIA, Oakland, CA (US)

(72) Inventors: Tammy Chang, San Francisco, CA (US); Maria Sekyi, San Francisco, CA (US)

(21) Appl. No.: 18/037,903

(22) PCT Filed: Nov. 19, 2021
(86) PCT No.: PCT/US21/60170

§ 371 Date: May 19, 2023

Related U.S. Application Data
(60) Provisional application No. 63/116,612, filed on Nov. 20, 2020.

(51) Int. Cl. C12M 1/00; C12M 3/04

(52) U.S. Cl. CPC C12M 23/02; C12M 23/22; C12M 27/10

(57) ABSTRACT

Articles of manufacture and associated methods in the field of tissue engineering are provided. Spherical vessels are provided that remove the detrimental mechanical features found in conventional culture vessels, promoting cell-to-cell interactions necessary for the formation of large functional tissues. The spherical vessels may employ structures that recapitulate blood vessels, providing a substrate for the growth of transplantable tissues and the formation of highly functional parenchyma with a microvascular network. Additionally, methods encompassing culture under simulated or actual microgravity are disclosed, wherein disruptive mechanical cues that confound the effective formation of large functional tissues are removed. By these systems and methods, highly vascularized and functional organoids, organs, and tissues suitable for transplantation may be produced in vitro.

### CROSS-REFERENCE TO RELATED APPLICATIONS

This application claims the benefit of priority to U.S. Provisional Patent Application Ser. No. 63/116,632 entitled “Tissue Orb,” filed Nov. 20, 2020, the contents of which application are hereby incorporated by reference.

### STATEMENT REGARDING FEDERALLY SPONSORED RESEARCH OR DEVELOPMENT

This invention was made with government support under grant number 16-16ROSBDFP-0030, awarded by the National Aeronautics and Space Administration and grant number 1830768, awarded by the National Science Foundation. The government has certain rights in the invention.

### BACKGROUND OF THE INVENTION

There is a strong need in the art for functional tissues to augment or replace failing, diseased, or damaged organs and functional tissues in the human body. By infection, deleterious mutations, trauma, oxidative damage, or aging, essential functional tissues in the body become damaged or dysfunctional, ultimately resulting in organ failure. Currently, the only effective therapy for end-stage organ failure is transplant, and a critical shortage of donor organs results in thousands of deaths each year, as well as the attendant societal problems of organ trafficking, transplant tourism and exploitation of vulnerable populations. Accordingly, there is a strong need in the art for functional tissue replacements.

Tissue culture and engineering can potentially fulfill the overwhelming medical need for replacement organs. In theory, by use of suitable cell populations and appropriate cues and culture conditions, functional tissues can be grown ex vivo and transplanted into subjects to augment or replace failing organs. Indeed, by current methods, cells in culture can be induced to self-assemble into small three-dimensional organoids that recapitulate some in vivo forms and functions, typically no larger than 500 μm in size. However there remains an immense bottleneck that has prevented clinical application of synthetic organs produced ex vivo: the lack of an effective strategy to produce a functional microvascular network within a thick tissue volume. There remains a need in the art for methods of creating of an interconnected microvasculature network amongst metabolically-active parenchymal cells, resulting in a large (for example, at scales of centimeters) macroscopic tissue construct that can be grafted onto a host blood vessel.

The systems disclosed herein provide the art with novel solutions for the problem of creating functional organ replacements. The inventions disclosed herein meet the aforementioned needs by the use of novel culture vessel designs and culture under low gravity or like conditions, promoting three-dimensional cellular self-organization of high-quality tissues.

### SUMMARY OF THE INVENTION

In a first aspect, the scope of the invention encompasses novel spherical tissue culture vessels. The inventors of the present disclosure have determined that standard culture vessel wells comprising corners, flat planes, and other such features will detrimentally affect tissue formation by creating preferential attachment sites and cues that interfere with tissue formation cues and cause distortions. By the use of spherical vessels, mechanical features that negatively affect tissue formation are removed and the cell-to-cell interactions necessary for the formation of large functional tissues are enabled.

In a second aspect, the scope of the invention encompasses novel culture vessels with structures that recapitulate blood vessels. As described below, the culture vessels of the invention utilize a central hollow conduit functionalized with angiogenic agents. This structure provides a substrate for the growth of transplantable tissues and the formation of highly functional parenchyma with a microvascular network that feeds into a readily grafted central macro blood vessel.

In a third aspect, the scope of the invention encompasses methods of culturing tissues under simulated or actual microgravity. The inventors of the present disclosure have determined that tissue culture under normal gravity creates mechanical cues that confound the effective formation of large, functional tissues. By culturing tissues under reduced gravity and using the novel vessels of the invention, disruptive cues are removed and cells are freed to self-organize and form critical microanatomical relationships that enable natural developmental paths as found in vivo.

### BRIEF DESCRIPTION OF THE DRAWINGS

FIGS. 1A, 1B, and 1C. FIG. 1A-1C depict an implementation of the invention wherein the spherical well is formed by joining two blocks of transparent material comprising hemispherical wells. FIG. 1A depicts a hemispherical block element of the invention, comprising a rectangular block of material comprising a hemispherical well or void. The block further comprises several channels, including a channel that will form the central conduit input, a channel that will form the central conduit output, a channel that will form an ancillary input channel, and a channel that will form an ancillary output channel. FIG. 1B depicts a first and second block, configured to be joined, and a central conduit. FIG. 1C depicts the joined blocks, forming a spherical well with central conduit and ancillary input and output channels.

FIGS. 2A, 2B, 2C, 2D, 2E, and 2F. FIG. 2A-2F depict an implementation of the invention wherein the spherical well is formed by joining two hemispherical bodies which form the tissue orb, wherein each hemispherical body is housed in a support block. FIG. 2A depicts a single hemispherical demiorb. FIG. 2B depicts a pair of demiorbs joined to form a spherical culture vessel. FIG. 2C depicts a single support block configured to house the hemispherical demiorb of FIG. 2A. FIG. 2D depicts the hemispherical demiorb when housed in the support block. FIG. 2E depicts a pair of demiorbs housed within support blocks configured to be joined around a central conduit. FIG. 2F depicts the two support blocks of FIG. 2E when joined to create a spherical well within (not visible).

FIGS. 3A and 3B. FIG. 3A depicts a central conduit of the invention, comprising a substantial tubular body. FIG. 3B depicts a central conduit of the invention ensheathed by a cell scaffold structure.

FIGS. 4A and 4B. FIG. 4A depicts a process for differentiation of a liver organoid or hepatic tissue by the devices and methods of the invention. FIG. 4B depicts albumin production by cells of self-assembled organoids produced under microgravity and conventional matrigel-grown organoids.

### DETAILED DESCRIPTION OF THE INVENTION

Part I. Tissue Orb

The novel culture vessels of the invention will be referred to herein as “tissue orbs,” based on the spherical wells thereof. A tissue orb, as used herein, encompasses any spherical culture vessel, i.e. a vessel wherein culture medium and cells may be incubated and wherein the vessel comprises a spherical or substantially spherical well or inner volume. As used herein, reference to “spherical wells” may encompass an inner volume comprising a void or space having a substantially spherical or spheroid geometry that lacks corners, edges, flat planes, and protrusions. In various embodiments, “spherical” may refer to a perfect sphere, imperfect sphere, spheroid, or ellipsoid, such as a flattened sphere or oblong spheroid. The tissue orbs of the invention. The wells are defined by spherical walls or a encasement.

Accordingly, in one embodiment, the scope of the invention encompasses a tissue culture system, wherein the tissue culture system comprises a tissue culture vessel, wherein the tissue culture vessel comprises: an inner volume having substantially spherical geometry lacking corners, edges, flat planes, and protrusions; and the tissue culture vessel further comprises two or more ports accessing the inner volume, wherein the two or more ports comprises a first port and a second port, and wherein the first port and second port are configured to receive the opposing ends of a conduit, as described below.

Channels Housing the Central Conduit. In a primary implementation, the tissue orb is utilized with a central conduit, as described below. In such implementations, the tissue orb will comprise two or more ports accessing the inner volume, wherein the two or more ports comprises a first port and a second port, and wherein the first port and second port are configured to receive the opposing ends of a conduit. Ports refer to openings in the outer wall of the tissue culture vessel by which the inner volume may be accessed, for example in fluid connection with outer elements.

In a primary implementation, the ports are configured as channels. In one embodiment, the culture vessel comprises a first conduit channel and a second conduit channel, wherein the first and second conduit channels are configured to receive or house the opposing ends of the central conduit. These channels serve as anchoring sites for the ends of the central conduit and also as input and output ports for the flow of medium through the central conduit. In one implementation, the channel is formed by complementary hemicylindrical channels that, when joined, form a circular tubular channel, as described in the exemplary embodiments below. Alternative geometries, such as square or irregular shaped channels may also be used. Alternatively, the central channel may be formed by drilling holes into the body housing the spherical well.

In a primary embodiment, the first and second central conduit channels are aligned at opposite poles of the spherical well, forming a linear pathway that transverses the geometrical center of the spherical well, in order to accommodate a linear central conduit. However, off-axis channels and non-linear configurations may be used as well.

Ancillary ports. The culture vessels of the invention may further comprises one or more ancillary ports in addition to the two conduit ports. The ancillary ports comprise openings in the well volume by which material may be introduced and/or withdrawn from the well, for example, growth medium. Accordingly, in one embodiment, the scope of the invention encompasses a culture vessel comprising a spherical inner volume and further comprising three or more ports, wherein the third or more port comprises an ancillary port accessing the inner volume of the tissue culture vessel. In one embodiment, the tissue culture vessel of the invention comprises one or more ports comprising an ancillary input port and one or more ancillary ports comprising an output port. In a primary implementation, the ports are in connection with elements for flowing material into and out of the inner volume of the tissue culture vessel.

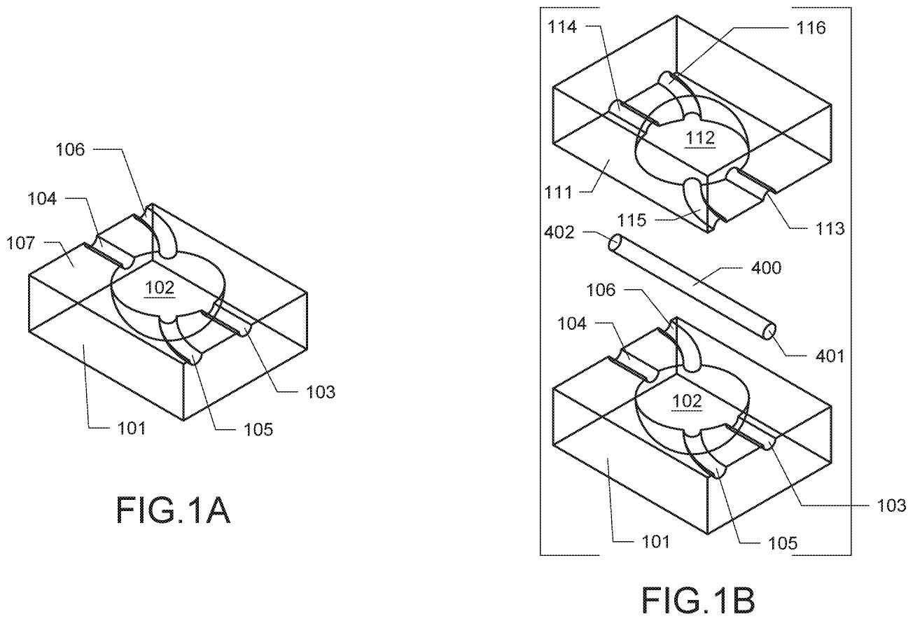

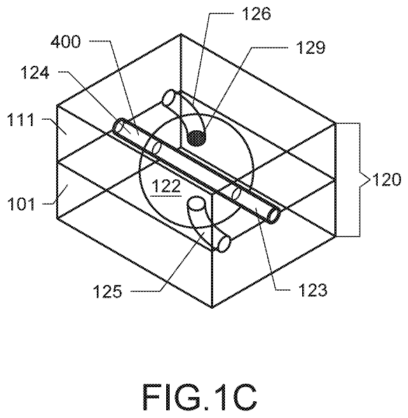

In a primary implementation, the ports are configured as channels. In one implementation, the tissue orb comprises a first ancillary channel comprising an input port and a second ancillary channel comprising an output port, by which ports growth medium or other materials may be introduced and withdrawn from the spherical well. For example, the tissue orb depicted in FIGS. 1A-C comprises two ancillary channels which may serve as input and output ports. In some embodiments, the output port is fitted with a screen, mesh, filter or other article that prevent the escape of cells and cell aggregates from the spherical well. For example, filter elements with a pore sizes in the range 1-100 μm may be used to prevent the loss of cells or organoids from the well. An exemplary filtering element 129 is depicted in FIG. 1C.

Tissue Orb Configurations. The tissue orbs of the invention may be configured as any number of different articles. In some implementations, the tissue orbs of the invention may be formed as a single piece or material. For example, in one embodiment, the tissue orb comprises a hollow spherical shell consisting of a single piece. In other implementations, the tissue orb is configured as an assembly of two or more pieces that are joined to create a tissue culture vessel with a spherical well. In a primary implementation, the tissue orb of the invention is formed by two or more complementary pieces that are joined to form a watertight spherical well. For example, in one implementation, two blocks comprising hemispherical indentations or voids are joined to form a body with a spherical well therein. In another implementation, two hemispherical objects (“demiorbs”) are joined to form a hollow sphere.

Illustrative Embodiment 1. An illustrative embodiment of the invention is depicted in FIGS. 1A-1C. In this implementation, the tissue orb is formed by joining two identical transparent pieces comprising hemispherical wells, wherein, once joined, the two articles form a solid block comprising a spherical well therein.

FIG. 1A depicts a block 101 comprising a flat surface 107 surrounding a hemispherical well or indentation 102. In this illustrative embodiment, multiple hemicylindrical (bisected cylinders) channels are present. A first 103 and second 104 hemicylindrical channel are present at opposite poles of the hemispherical well. These opposing channels will, upon assembly, form a first cylindrical channel and second cylindrical channel wherein these first and second channels house opposing ends of the central conduit, as described below. In this illustrative embodiment, the block further comprises additional hemicylindrical channels: a first ancillary hemicylindrical channel 105 that, when assembled with a complementary channel, will form an input channel in connection with the spherical well; and a second ancillary hemicylindrical channel 106 that, when assembled with a complementary channel, will form an output channel in connection with the spherical well.

FIG. 1B depicts two blocks 101 and 111, the two blocks being identical and being arranged to be joined around a central conduit 400. The length of the central conduit 300 is greater than the diameter of the hemispherical wells 102 and 112 of blocks 101 and 111, such that each end 401 and 402 of the central conduit will protrude from the spherical well and will be encased or sandwiched between within the channels 123 and 124, formed by sandwiching channels 103 and 104 of block 101 with channels 113 and 114 of block 111.

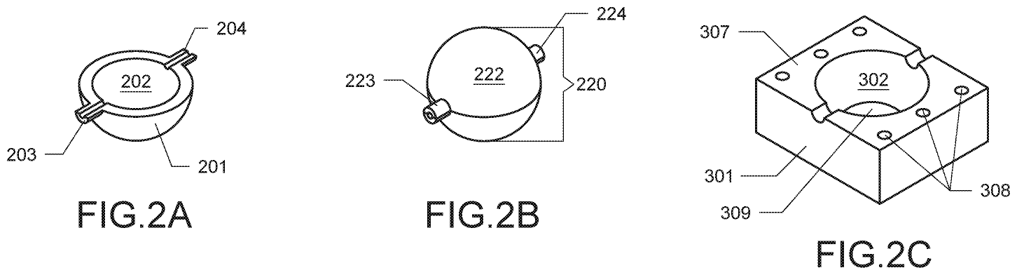

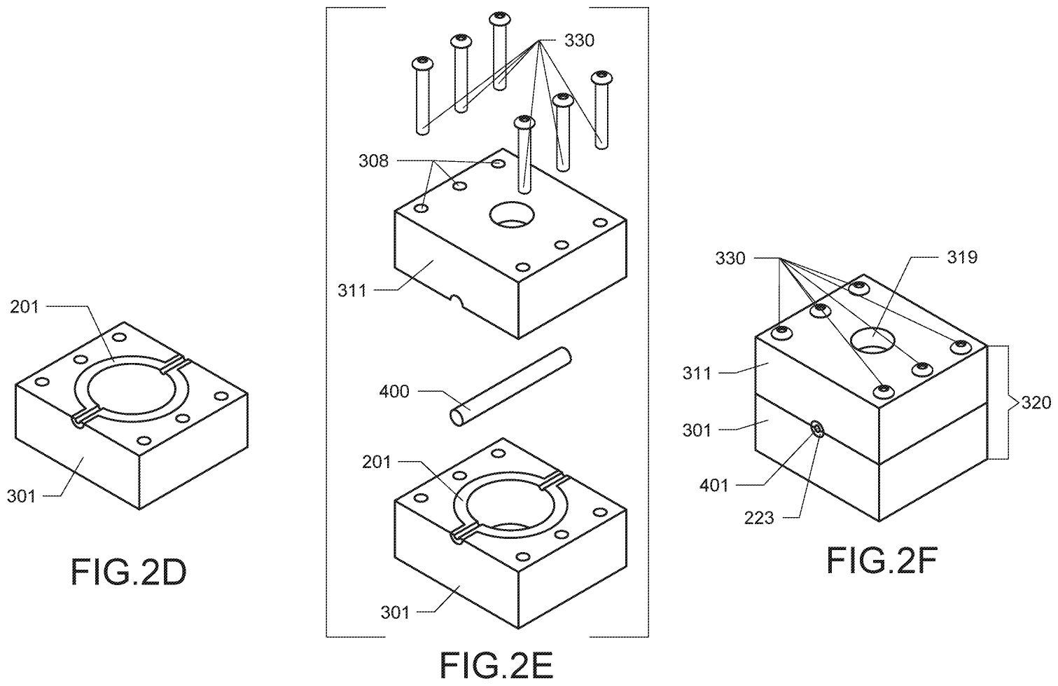

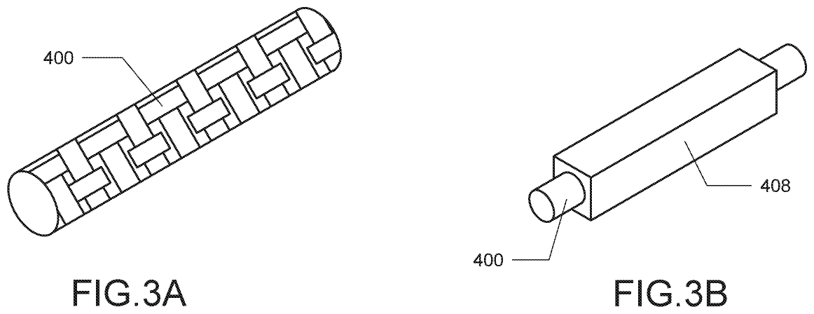

FIG. 1C depicts the two blocks 101 and 111 of FIG. 2B wherein the blocks have been aligned symmetrically and joined at surfaces 107 and 117 to form a single assembly 120. The assembly comprises a central spherical well 122. The assembly further comprises a first conduit channel 123 and a second conduit channel 124, each comprising a lumen in connection with the spherical well 122 and extending to the border of the assembly 120, wherein the conduit channels are aligned on opposing poles of the spherical well 122, enclosing the central conduit 300. The assembly further comprises a first ancillary channel 125 serving as an input port to the spherical well 122 and a second ancillary channel 126 comprising an output channel from the spherical well 122, by which input and output channels, medium and other materials may be introduced to the spherical well.

Illustrative Embodiment 2. An alternative implementation of FIG. 2A-2E depicts an alternative implementation of the tissue orb. In this implementation, the tissue orb is formed by joining two hemispherical demiorbs to form a hollow spherical vessel comprising a spherical well therein.

FIG. 2A depicts a single demiorb comprising a spherical shell 201 defining a hemispherical space 202. Protruding from the shell are a first hemicylindrical tube 203 and a second hemicylindrical tube 204, located at opposing poles of the demiorb.

FIG. 2B depicts two demiorbs 201 and 211 joined to form a spherical orb 220 comprising a spherical well 222 therein. The orb of FIG. 2B further comprises a first cylindrical tube 223 and a second cylindrical tube 224 protruding at opposing poles of the spherical shell, formed by the union of first hemicylindrical tube 203 and a second hemicylindrical tube 204 of block 201 and first hemicylindrical tube 213 and a second hemicylindrical tube 214 of block 211.

In the illustrative embodiment depicted in FIG. 2A-2F, the demiorbs are each housed in a support block to facilitate their alignment and union. FIG. 2C depicts a support block 301 comprising a rectangular body having a flat upper surface 307 with hemispherical depression 302 or cavity configured to receive a demiorb. The support block further comprises holes 308 for receiving fasteners 330. The support block further comprises an opening comprising a viewing port 309 for accessing the spherical well by imaging means. FIG. 2D depicts a demiorb 201 sitting within a support block 301.

FIG. 2E depicts two support blocks 301 and 311, each housing a demiorb 201 and 211, configured to be joined around a central conduit 400.

FIG. 2F depicts the final assembly of the two support blocks 301 and 311, joined by use of fasteners 330 to form assembly 320. The support blocks house two demiorbs aligned and joined to form a spherical well (not visible) accessed by a first channel 232 and second channel (not visible) which channels house the opposing ends (401 and 402, not visible) of the enclosed central conduit 400. The enclosed orb 221 may be viewed through port 319 of the support block.

Watertight Seal. In the tissue culture vessels of the invention, assemblies forming the spherical well may be joined by any suitable means. Blocks or support blocks may comprise holes for receiving fasteners such as screws or bolts, and may be joined by use of such fasteners, for example, as in FIGS. 2E and 2F. Alternatively, blocks may be joined by clamping them together with clamping elements. The two or more pieces must be joined in a substantially waterproof seal in order to retain liquid medium. Waterproof seals may be achieved or enhanced by use of sealants such as silicone, cyclic olefin copolymers, polymethacrylate, polystyrene, and other sealants known in the art. Alternatively, a gasket comprising rubber or other polymeric material may be disposed at the borders of joined pieces. Preferably, the union is readily reversible so that contents of the culture vessel well may be retrieved.

Tissue Orb Materials. The body of the tissue orb comprising the spherical well may comprise any suitable material for cell culture. Materials to which cells do not readily adhere are preferred. Exemplary materials include, for example, glass, silicone, metals, polystyrene, polycarbonate, polypropylene, polyacrylamide, poly methyl methacrylate (PMMA), polydimethylsiloxane (PDMS), cyclo-olefin copolymer, or other such materials known in the art.

In a primary embodiment, the tissue orb is made of a transparent or translucent material to facilitate imaging of the well of the tissue orb. For example, PDMS is substantially transparent.

In a primary implementation, the material defining the spherical well is made of a suitable cell culture material. In an alternative embodiment, the material defining the spherical well comprises a first, base material that provides structural support and which defines the contours of the spherical well, wherein the well surface is coated with a second cell culture material to impart the desired properties to the well surface, such as biocompatibility and prevention of cell adhesion.

In some embodiments, the tissue orb is housed in a support block, for example, as depicted in FIG. 2A-2F. In such implementations, the support block may comprise any material. Exemplary materials include polymeric materials such as polypropylenes or polycarbonate, or metals such as aluminum or titanium. In some embodiments, the support block is made of a transparent or translucent material to facilitate imaging of the well of the tissue orb. In one embodiment, the support block comprises one or more openings through which the enclosed spherical well may be imaged. The support block may further comprise fittings or other structures to facilitate connection to microfluidic elements such as fittings for tubing.

The components of the tissue orb may be fabricated by standard techniques such as molding, machining, stamping or printing to create hemispherical shells or wells of the desired size and shape and having other features such as access channels for the central conduit and ports for microfluidics.

Size and Scale. The culture vessels of the invention may advantageously be scaled across a wide range of sizes. For example, the diameter of the spherical culture well may range from 1 mm to 100 mm or larger, for example, 5 mm, 10 mm, 20 mm, 50 mm, 75 mm, 100 mm, or greater. In an experimental context, smaller culture vessels may be used to grow experimental tissues for drug testing or other applications. In a therapeutic context, larger culture vessels may be used, for example, for the formation of transplantable tissues, for example, vessels having well diameters in the range of 10 to 100 cm.

Central Conduit. In a primary implementation, the tissue orb is utilized in combination with a central conduit. The central conduit is a substantially tubular article that comprises a blood vessel or a synthetic structure that recapitulates the structure and function of a blood vessel. In some implementations, the central conduit serves as a substrate upon which cultured cells will coalesce to form an organized tissue. The central conduit may further provide nucleation sites for a branched vascular network in the growing organoid. By the flow of growth media through the central conduit, the central conduit will also act as a nutrient source for the growing tissue, promoting the vascularization thereof. The central conduit may comprise a tubular structure that extends across the diameter of the spherical well into opposing conduit channels, for example, channels 123 and 124 as depicted in FIG. 1C.

In one implementation, the central conduit comprises a biological material such as a blood vessel, for example, an explanted blood vessel or a synthetic blood vessel made by tissue culture. In one embodiment, the explanted blood vessel comprises a stent or other support body to provide structure thereto.

In a primary embodiment, the central conduit comprises an article of manufacture configured to recapitulate the structure and function of a blood vessel. The central conduit will typically comprise a biocompatible, microporous material that facilitate exchange of materials between the solution flowing through the lumen of the conduit to the cells growing on and around it. In a general implementation, the central conduit, or a portion thereof, and surrounding tissue grown thereon is transplanted into the body of a recipient, as described below. Accordingly, the central conduit will generally comprise a biocompatible material suitable for long term implantation in the body, or a biodegradable and/or bioresorbable material.

In one embodiment, the conduit comprises a medical textile such as a mesh, for example, a woven, braided, or other porous textile. Exemplary conduit materials include polyethylene terephthalate (Dacron™), polyglycolide (PGA), polylactide (PLA), poly-lactic-co-glycolide (PLGA), polyacrylonitrile, polytetrafluoroethylene (PTFE), polycaprolactone, poly ethylene glycol diacrylate (PEGDA), poly ethylene glycol dimethacrylate (PEGDMA), gelatin methacrylate (GelMA), microspun silk, and other materials used in the art for medical implants. Mesh pore sizes may vary, being selected for optimal exchange of materials between the conduit interior and the cells growing on the outer surface. Exemplary pore sizes or porosity values are, for example, in the range of 0.5-100 μm.

In a primary implementation, the outer surface and/or, optionally the inner surface, of the conduit is functionalized with one or more biologically active molecules. In a primary embodiment, the one or more biologically active molecules is a pro-angiogenic agent. Exemplary pro-angiogenic agents include vascular endothelial growth factor (VEGF), VEGF mimetics, periostin, chondroitin sulfate methacrylate, fibroblast growth factor-2 (FGF-2), platelet derived growth factor (PDGF), platelet derived endothelial cell growth factor (PD-ECGF), angiopoietins (Ang), hepatocyte growth factor (HGF), insulin like growth factors (IGFs), and tumor necrosis factor (TNF). Biologically active molecules may be deposited at any concentration, for example, in the range of 10 ng/ml to 100 mg/ml.

The conduit surface may comprise additional biomolecules that facilitate tissue nucleation or which promote the desired cellular differentiation processes, for example, extracellular proteins including and not limited to collagens, elastins, laminins, fibronectins, entactin, hyaluronan, glycosaminoglycans, and proteoglycans. In some implementations, the central conduit is functionalized with lipids, carbohydrates, or other biomolecules.

The biomolecules may be conjugated to the outer surface and/or inner surfaces of the conduit, for example, by conjugation chemistries known in the art. Alternatively, the biomolecules may be provided in drug eluting materials formulated for continuous release of agents that are coated onto the conduit surfaces or infused into the conduit material. Exemplary materials include polymers or hydrogels, such as hydrogels made from collagen, polyethylene glycol (PEG), poly ethylene glycol diacrylate (PEGDA), gelatin methacrylate (GelMA), hyaluronic acid, agarose, methylcellulose, and other materials.

In a primary implementation, a single, tubular conduit is utilized. It will be understood that the scope of the invention encompasses other configurations, such as the use of two or more conduits, branching conduits, and other arrangements that recapitulate various blood vessel architectures.

Cellular Scaffolds. In some implementations the central conduit further comprises one or more structures comprising a cellular scaffold. For example, cellular scaffold may be formed around the central conduit or otherwise joined thereto. The cellular scaffold may achieve various functions, including, providing a nucleation site for cultured cells, guiding or defining the form of a growing organoid, or providing a source of cells for the formation of organoids. The cellular scaffold will comprise a matrix material and may further comprise cells seeded therein.

The cellular scaffold may comprise any selected configuration, including a spherical configuration, irregular configuration, or the shape of an organ to be replicated by the cultured organoid. In some embodiments, the scaffold formed by molding. In some embodiments, the scaffold comprises a templating structure that holds the matrix material in the desired shape.

Exemplary matrix materials include porous materials suitable for seeding with cells. Exemplary materials include: collagen materials such as reconstituted Type 1 collagen sponges or microfibrillar collagen sponges or hydrogels; proteoglycans; alginate-based substrates; chitosan; polycaprolactone; ceramic/collagen composite scaffolds; polymeric materials such as polyurethane scaffolds synthesized from lysine-di-isocyanate; plantbased matrix materials, for example cellulose, hemicellulose, and/or lignin; and admixtures of the aforementioned materials.

In some embodiments, the scaffold is seeded with cells prior to culture. For example, the scaffold may be incubated in a suspension of cells, prior to initiating culture. Cells may be seeded at any suitable density, for example, in the range of 10,000 to 100,000 cells per mm³ of matrix material, for example in the range of 20,000 to 40,000 cells per mm³ matrix material. For example, cellular scaffolds may be loaded with a selected number of cells, for example, between 1 and 10 million cells depending on the size of the scaffold and selected density of cells.

Culture systems of the invention. The tissue orbs of the invention may be utilized in a culture system. The culture systems of the invention will comprise a combination of devices for facilitating the culture of cells in the tissue orb, facilitating sampling and imaging of cultured cells in the tissue orb, and in some embodiments, providing microgravity environments for the growth of cells.

In use, the central conduit of the Tissue Orb will be in fluidic connection with external elements that will flow culture medium solution through the central conduit. Culture system elements may include: tubing connecting the ports or channels of the tissue orb with reservoirs of liquid medium; connection means for attaching tubing; pumps; flow regulators; sampling devices; and other components of fluidic systems.

Liquid medium may be selected based on the type of cells used and the target tissues being formed. Examples of growth medium include: Dulbecco's Modified Eagle Medium (DMEM), Eagle's Minimal Essential Medium (EMEM), RPMI, Ham's F-12, mTeSR(TM) (Gibco), Hepatocyte Culture Medium(TM)(Lonza Biosciences), Endothelial Basal Medium(TM) or Endothelial Basal Medium-2(TM) (Lonza Biosciences), or other media known in the art. These media may include additional supplements and factors, including nutrient molecules, minerals, hormones, small molecule agonists or inhibitors, growth factors, and cytokines/chemokines.

The culture systems of the invention may comprise a first liquid medium which is introduced to the well of the culture vessel, in which the central conduit is immersed. The culture environment within the spherical well may be controlled by input and withdrawal of culture medium via one or more ancillary ports, for example, an input ports an output port, for example, configured as channels that connect the spherical well space with external microfluidic or microfluidic elements, e.g. pumps, reservoirs of liquid media, waste collection vessels, regulators, analytical devices, and sampling devices. By these input and output channels, fresh media may be infused into the culture environment, and bioactive molecules may be introduced into the extracellular environment of the growing cells, for example, growth factors, nutrients, drugs, and other agents to promote or manipulate development and function of the tissue. In one embodiment, liquid media in the well is gradually and gently exchanged over a period of 1-3 days. In some implementations, the first liquid medium is a differentiation medium. Different types of differentiation medium may be introduced sequentially to drive differentiation of cellular structures in the vessel.

In some embodiments, the liquid medium that is flowed through the central conduit may comprise the first liquid medium of the culture well. Alternatively, the liquid medium that is flowed through the central conduit may comprise a second liquid medium composition. In one embodiment, the composition of the first liquid medium is selected to recapitulate aspects of the extracellular environment of differentiating tissue and the second liquid medium is selected to recapitulate aspects of the blood.

With regards to the liquid medium that is flowed through the conduit, or second liquid medium, it may be flowed through the central conduit by elements comprising a source of liquid medium (reservoir) and additional elements (e.g. pumps, flow regulators, etc.) for flowing culture medium through the conduit. Solution may be flowed through the conduit at physiologic rates and pressures found in vivo for corresponding organs. For example, the systems of the invention may be configured for flow rates that impart physiological shear stress (3-5 dyn/cm²) which assists in the formation of tight junctions and establishment of the vascular barrier, or increased shear stress (10-15 dyn/cm²) which promotes angiogenic sprouting. Different rates of flow may be used during the different stages of the culture process. In some implementations, the flow is regulated such that it is intermittent at the beginning of the culture period, progressing to continuous flow toward the end of the culture period.

The culture systems of the invention are configured such that the inner volume of the culture vessel well may be imaged. In such implementations, the walls of the spherical well will comprise a transparent or optically transmissive material, or will comprise one or more windows composed of such material. Support blocks may comprise optically transmissive material or may comprise openings by which the tissue orb can be accessed by imaging components. In some implementations, the culture systems of the invention comprise integrated imaging devices. Imaging devices include microscopy devices, for example, s devices configured for real-time live-cell time-lapse light microscopy, fluorescence microscopy and/or other types fluorescence microscopy.

Part II. Culture Under Reduced Gravity.

The scope of the invention encompasses methods for the formation of vascularized cell culture products, such as vascularized macroscopic tissues and organoids. The culture process by which the cell culture products are formed may be performed under normal gravity.

However, in a primary implementation of the invention, a portion or the entirety of the culture process in the tissue orb proceeds under conditions of reduced gravity. The inventors of the present disclosure have determined that under normal gravity, constant settling of cultured cells disrupts the biochemical cues that enable formation of thick tissue with high vascularization. In contrast, when cultured under reduced gravity, coalescing and growing tissues remain suspended, promoting stochastic cell-to-cell interactions and interactions with the central conduit blood vessel or blood vessel mimic, enabling in vivo-like development. Under reduced gravit, conditions of laminar flow with low turbulence, low shear stress, and 3-dimensional spatial freedom are created, advantageously enabling cells to self-associate.

As used herein, “reduced gravity conditions” encompasses any operational environment wherein the force of gravity is attenuated, negated, or absent. In various embodiments, reduced gravity conditions means conditions equivalent to less than 1 g of gravitational force, for example, 0.9 g, 0.8 g, 0.7 g, 0.6 g, 0.5 g, 0.4 g, 0.3 g, 0.2 g, 0.1 g or less than 0.1 g. In one embodiment, reduced gravity means zero-gravity or weightlessness, i.e., a zero average gravity or free falling condition.

Simulated Microgravity. In a first implementation, the culture methods of the invention are carried out under reduced gravity conditions comprising simulated microgravity. Simulated microgravity may be achieved by the use of devices configured to recapitulate a state of reduced gravity, for example, simulated microgravity with an average gravity vector of 0 g.

In a first embodiment, the device comprises a rotating wall vessel (RWV). The RWV is a rotating bioreactor wherein constant rotation of the fluid within the reactor creates a continuous state of falling for the contents, attenuating the effects of gravity. Exemplary RWVs include high-aspect rotating vessels (HARVs) and slow-turning lateral vessels (STLVs).

In another implementation, reduced gravity is achieved by the use of a random positioning machine (RPM), as known in the art. Random positioning machines encompass culture vessels mounted within two concentric frames, such that the vessel may be rotated around two independent axes, enabling rotation in continuously changing orientations and complex 3-dimensional vectors, such that the gravity vector of the vessel interior averages to zero, negating the effects of ambient gravity.

The scope of the invention further extends to any other devices that can attenuate or remove the influence of gravity, including clinostats and magnetic levitation devices.

Operation in Space. In another implementation, the Tissue Orb is operated in space, i.e. in orbit or otherwise away from the surface of the Earth. For example, in one embodiment, the culture process is performed at zero-gravity on a platform orbiting the Earth, such as the International Space Station, satellite, or other orbiting spacecraft. A significant gravitational force is still present at the orbiting altitude of human-made satellites. For example, at the orbiting altitude of the International Space Station, about 409 km, Earth's gravitational force is 0.9 g. However, orbiting craft experience continuous free fall, effectively creating the condition of weightlessness or zero-gravity. In one embodiment, the culture process is performed on an extraterrestrial body having lower gravity than Earth, for example, a lunar base (0.167 g).

Part III. Methods of Use and Tissue Production

The scope of the invention encompasses methods of producing organoids and other tissue culture products from progenitor cells In a general implementation, the method of the invention may encompass a process substantially as follows:
progenitor cells suitable for the formation of a selected cell culture product are introduced to the spherical inner volume of a culture vessel of the invention, wherein the culture vessel is filled with a first liquid medium;
flowing a second liquid medium through the central conduit; and
performing the sequential introduction of selected biomolecules into the first liquid medium of the spherical volume of the culture vessel and/or the second liquid medium flowed through central conduit;
wherein the sequential introduction of selected biomolecules is performed in a protocol suitable for the formation of the selected cell culture product; and
wherein a cell culture product comprising a macroscopic vascularized multicellular body is produced.

The scope of the invention will be understood to encompass variations of the enumerated method, including the performance of additional steps or performance of the steps in a different order.

Tissue Culture Products. The “tissue culture products” produced by the methods of the invention may encompass any of organoids, tissues, organs, and other multicellular articles. The methods of the invention may advantageously be utilized to produce macroscopic, vascularized multicellular structures, comprising tissues or tissue like multicellular bodies that are organized and which have a defined vascular system comprising one or well defined blood vessels and/or capillary beds. In one embodiment, vascularity is assessed by CD31 staining and the observation of organized vessels. In one embodiment, vascularity is assessed by immunohistochemical estimates of microvessel density (MVD). For example, MVD values exceeding a selected threshold may be used to determine vascular status, for example, scores of greater than 5 microvessels per mm², greater than 10 microvessels per mm², greater than 20 microvessels per mm², greater than 30 microvessels per mm², greater than 50 microvessels per mm², or greater than 100 microvessels per mm² may be used as thresholds indication sufficient vascularization.

In one embodiment, the vascularized cell culture product is a macroscopic multicellular structure of sufficient vascularity wherein, if grafted onto a blood vessel of a living subject, the structure will persist and/or function in the body. In one embodiment, the vascularized cell culture product is a macroscopic multicellular structure suitable for transplantation into a living subject. In one embodiment, the vascularized cell culture product is a functional multicellular structure, for example, capable of performing one or more biological functions of a native tissue such as lung, cardiac tissue, liver tissue, kidney tissue, muscle tissue, brain tissue or others.

The cell culture products of the invention may comprise organoids, tissues, and other structures of any size. In a primary implementation, the cell culture products are macroscopic. Exemplary sizes of cell culture products may be, for example, in the range of 100-500 μm in size, larger structures of 500 μm to 5 mm in size, and larger structures in the range of 5 mm to 5 cm or greater in size.

Culture methods. In one implementation, the scope of the invention encompasses a method of producing a macroscopic vascularized multicellular structure by the culture of cells under conditions of reduced gravity, for example, by the use of any tissue culture vessel. In this implementation, culture under conditions of reduced gravity encompasses imposition of reduced gravity conditions on the cultured cells for any portion of, or for the entirety of the culture process. In various embodiments, the imposition of reduced gravity conditions may be applied by a device for achieving simulated microgravity, for example, any of a rotating wall vessels, random positioning machines, clinostats, and magnetic levitation devices. In other embodiments, the imposition of reduced gravity conditions is achieved by culturing the cells in space or on an extraterrestrial body having lower gravity than Earth, e.g., the moon.

In one implementation, the scope of the invention encompasses a method of producing a macroscopic vascularized multicellular structure by the culture of cells within a spherical or substantially spherical tissue culture vessel. In some embodiments, the spherical or substantially spherical tissue culture vessel comprises a central conduit upon which the cultured cells are deposited or aggregate.

In another implementation, the scope of the invention encompasses a method of producing a macroscopic vascularized multicellular structure by the culture of cells utilizing a spherical or substantially spherical tissue culture vessel wherein reduced gravity conditions are imposed on the growing cells for a portion or the entirety of the culture process. In various embodiments, the imposition of reduced gravity conditions may be applied to the spherical culture vessels of the invention by a device for achieving simulated microgravity, for example, any of a rotating wall vessels, random positioning machines, clinostats, and magnetic levitation devices, or by performing a portion or the entirety of the culture process in space or extraterrestrial body.

In a general process of the invention utilizing a spherical tissue orb of the invention, the following steps are performed: a central conduit is introduced to a spherical vessel or otherwise encased therein by joining two hemispherical elements around the central conduit; wherein the central conduit comprises a cellular scaffold seeded with cells and/or, a population of progenitor cells is introduced to the spherical vessel; initiation of growth media flow through the central conduit; and, optionally culturing under reduced gravity conditions.

Tissue development may be promoted by a series of manipulations in the culture conditions to promote their formation, as known in the art. Upon formation of functional organoids or tissues with an established microvascular system, the tissue orb may be opened to extract the formed tissue for experimental or clinical uses.

Progenitor cells may comprise pluripotent cells, or differentiated cells derived from pluripotent cells such as embryonic cells or induced pluripotent stem cells. Primary adult cells derived from culture, or from donors, including autologous donors, may be used as well. In one embodiment, tissue explants are utilized. The selected mixture of progenitor cells will be based on the desired end product, by protocols known in the art. For example, in the formation of liver tissues, a mixture of human induced pluripotent stem cell (iPSC)-derived hepatocytes, umbilical vein endothelial cells (HUVECs), and mesenchymal stem cells (MSCs) may be utilized to provide precursors for the vasculature, hepatocytes, and extracellular matrix found in the mature organoid. Cells may be introduced as single cells, cell aggregates, organoids, tissue explants, mixtures of the foregoing, or in any other form.

The progenitor cells may be introduced to the central conduit and/or the spherical culture well prior to sealing the vessel, may be introduced subsequent to sealing via the input channel, or may be provided in cell-loaded scaffold structures adhered to the central conduit. Cells may be seeded at any suitable density, for example, in the range of 10,000 to 500,000 cells per ml liquid medium, for example in the range of 20,000 to 100,000 cells per ml liquid medium.

The scope of the invention encompasses the formation of any type of tissue. For example, tissues of the following types may be produced: liver, kidney, pancreatic, prostate, lung, cardiac, thyroid, intestinal, mammary, prostate, brain (e.g. hippocampal, cerebellar, cerebral, optic, etc.), muscle, pancreas, dermal, and any other tissue, organ, or organoid types known in the art.

The culture media will be selected based upon the selected tissue to be formed. Exemplary culture media include standard growth and differentiation media known in the art. Culture media composition may be manipulated in a series of steps to promote the directed differentiation and growth of the tissue. For example, growth factors or chemical cues may be added to and withdrawn from the growth media based on elapsed time or the achievement of developmental milestones. Such factors may be introduced via the solution flowing through the central conduit or to the extracellular environment by the culture well input port. Development of the tissue may be monitored visually, or by the appearance of markers in cellular exudates sampled by means of the output port. Alternatively, cellular material may be sampled by biopsy instruments introduced via an ancillary port comprising a sampling port, for example, needles, punches, or other sampling instruments, for example, introduced on endoscopic catheters and the like.

Culture conditions may be further manipulated to promote tissue formation by modulating the flow rate of the medium through the central conduit, or the flow rate of media through the extracellular environment via the well input and output ports. Flow may be intermittent, or continuous. In one embodiment, flow through the central conduit is intermittent early in organoid formation, and transitions to continuous flow as initial tissue structures coalesce and differentiate into thicker, functional tissues.

In some cases, culture is performed under normal gravity. If reduced gravity is applied, such reduced gravity condition may be imposed continuously, or intermittently. For example, in some implementations, the entire duration of the culture process is performed under conditions of reduced gravity. In other implementations, reduced gravity is imposed during certain developmental windows. In some implementations, the magnitude of gravity reduction is tapered near the end of the culture process to acclimate the tissues to ambient 1 g gravity.

The culture process is completed upon formation of a vascularized multicellular structure having one or more selected attributes. In one embodiment, the selected attribute is the appearance of one or more developmental biomarkers. The liquid culture medium of the well, or that flowed through the central conduit, may be sampled, for example, by an ancillary output port and then assayed for the presence of biomarkers indicative of the desired developmental endpoint. In some embodiments, the selected attribute is a functional attribute, wherein the liquid medium and/or the multicellular structure are assayed for functional abilities, e.g. metabolic, enzymatic, or other biological activity of the selected tissue type. The multicellular structure may be assayed for example by removing a biopsy or explant thereof via a sampling port in the well. In some embodiments, the selected attribute is size, wherein the organoid or tissue is harvested from the culture vessel upon reaching a selected size, e.g. assessed by diameter, volume, or other dimension.

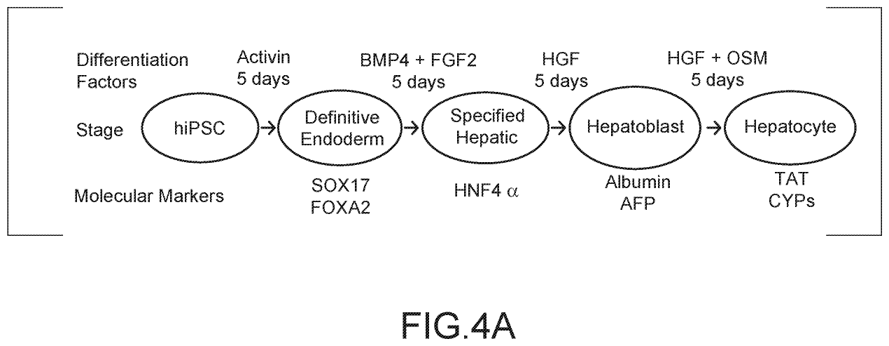

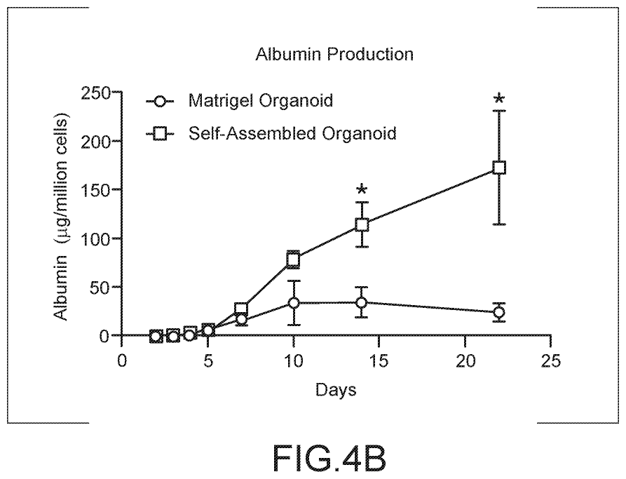

Liver Organoid Formation. In one implementation, the vessels, systems and method of the invention are utilized for the formation of liver organoids or tissues, by protocols known in the art. In one embodiment, the differentiation protocol is implemented according to the following sequence, as depicted in FIG. 4A:
Stage 0: The progenitor cells are introduced to the tissue orb. In one embodiment the progenitor cells are supplied in a cellular scaffold formed around the central conduit. The progenitor cells may comprise pluripotent cells or a mixture of cell typess. In one embodiment, the progenitor cells comprise a mixture of induced pluripotent stem cells, umbilical vein endothelial cells, and mesenchymal stem cells;
Stage 1: the progenitor cells are initially grown in media supplemented with activin;
Stage 2: after about five days, and/or upon the formation of a definitive endoderm and/or significant expression of the transcription factors SRY-box 17 (SOX17) and forkhead box 2 (FOXA2), activin is withdrawn and the media is supplemented with bone morphogenetic protein (BMP) and fibroblast growth factor (FGF);
Stage 3: after about five days, and/or upon the observation of significant hepatic and/or significant expression of hepatocyte nuclear factor 4α (HNF4α), BMP and FGF are withdrawn and the media is supplemented with hepatocyte growth factor (HGF);
Stage 4: after about five days, and/or upon the observation of hepatoblasts, and/or the significant expression of alpha fetoprotein (AFP) and albumin, the HGF medium is further supplemented with oncostatin M (OSM);
Stage 5: after about five days, and/or the observation of hepatocyte cells, and/or the significant expression of tyrosine aminotransferase (TAT) and cytochrome P450 enzymes (CYPs), the organoid may be allowed to grow and mature further or may be withdrawn from the Tissue Orb for transplantation or other uses.

Alternative Implementations. In an alternative implementation, the reduced gravity tissue culture process is performed using any tissue culture vessel, for example, a cuture vessel comprising a non-spherical well or inner volume. In this implementation, any type of culture vessel having a well of any geometry may be utilized. In this implementation, the reduction of gravity on the cultured cells during a portion or entirety of the culture process is sufficient to enable the production of vascularized cell culture products regardless of culture well geometry. In an exemplary implementation, the general method comprises a method of producing a vascularized cell culture product, comprising
introducing progenitor cells suitable for the formation of a selected cell culture product to a culture vessel containing a liquid medium;
performing the sequential introduction of selected biologically active molecules into the liquid medium of the culture vessel;
wherein the sequential introduction of selected biologically active molecules is performed in a protocol suitable for the formation of the selected cell culture product;
wherein a cell culture product comprising a macroscopic vascularized multicellular body is produced; and
wherein the cells within the tissue culture vessel are subjected to a state of reduced gravity or weightlessness during a portion or the entirety of the culture process.
   The foregoing method may optionally employ a central conduit through which liquid medium is flowed, configured as described above for culture methods in spherical vessels.

In some implementations of the invention, the culture process is performed utilizing spherical culture wells but without the use of a central conduit. In some cases, aggregation of cultured cells into organized vascularized multicellular structures is achieved without a blood vessel or blood vessel mimic. Such culture may be at normal gravity or with reduced gravity on the cultured cells during a portion or entirety of the culture process.

Part V. Uses of Tissues Produced by the Methods of the Invention.

In some embodiments, the tissue produced by the devices or methods of the invention comprises a multicellular structure suitable to be transplanted into a subject. Such tissues can be surgically implanted by means known in the art, under conditions or by techniques that promote vascular connection between the host and implanted tissue. In some embodiments, a host blood vessel is grafted to the central conduit or other vascular elements of the tissue. In such embodiments, the central conduit structure remains in the tissue and is grafted to a blood vessel in the subject. In such implementations, the central conduit will comprise a biocompatible material suitable for long term implantation in the body, or a biodegradable and/or bioresorbable material.

In a primary embodiment, the recipient is a human subject in need of an organ transplant. In some embodiments, the recipient is a non-human animal comprising a veterinary subject, such as a pet or livestock. In some embodiments, the recipient is a test animal. In some embodiments, the tissue comprises human cells which are introduced into a test animal as a xenograft, for example, a test animal comprising an immune-compromised model suitable for xeno-transplanted cells.

In some embodiments, the tissue produced by the methods and devices of the invention comprises an experimental platform for investigating the effects of different treatments on tissue development as a proxy of a native organ. In these implementations, the tissue can be exposed to different agents during or after tissue formation to test the effects of such agents on various parameters, such as toxicology, physiology, function, or other factors. Agents of interest may encompass any composition, such as a drug, drug candidate, growth factor, ligand, hormone, immune factor such as a cytokine or chemokine, potential toxin, pollutant, pathogen, or immune cell. In one embodiment, agents of interest are introduced at the onset of tissue formation and/or during organ formation, for example by infusing the central conduit with the selected agent, for example, in drug-eluting compositions such as a hydrogel. In another implementation, the agent is provided to the developing tissue via the medium flowing through the conduit to mimic agent delivery via the circulatory system. In some implementations, test agents are provided to the extracellular medium of the culture well via the input port. In some embodiment, the agents are introduced to the tissue after its formation and extraction from the culture vessel.

In some embodiments, the tissue comprises a disease model, comprising tissue with mutations or induced dysfunctions. The tissue can serve as a platform for observing pathological processes or testing therapeutic interventions.

In one aspect, the scope of the invention encompasses a product produced by the method of the invention. In one implementation, the scope of the invention encompasses a cell culture product produced by the use of a culture vessel comprising a spherical well. In one implementation, the scope of the invention encompasses an cell culture product produced by the use of a culture vessel comprising a spherical well and a central conduit disposed therein through which liquid medium is flowed. In one implementation, the scope of the invention encompasses an cell culture product produced by culturing cells under conditions of reduced gravity. In one implementation, the scope of the invention encompasses an cell culture product produced by the use of a culture vessel comprising a spherical well under conditions of reduced gravity. In various implementations, the product-by-process is a multicellular vascularized structure, such as an organoid, tissue, or organ, for example, selected from the group consisting of

### EXAMPLES

### Example 1. Generation of Hepatic Organoids within the Tissue Orb

A tissue orb with spherical inner well having a 2 cm diameter was assembled sterilely in a biosafety cabinet and loaded with HepG2 human hepatocellular carcinoma cell line at 50,000 cells/ml liquid medium density. The Tissue Orb was secured to a Random Positioning Machine (RPM) and cultured under conditions of simulated microgravity (average gravity vector 0-g) within an incubator at 37° C. and 5% CO₂. After 3 days, self-assembled HepG2 organoids, ranging 100-200 m in size, were formed within the Tissue Orb and visualized by brightfield microscopy. This example demonstrated that culture within the Tissue Orb in microgravity created an environment conducive to the self-assembly of organoids and tissues.

### Example 2. Attachment of Mesenchymal Stem Cells (MSCs) to the Tissue Orb Central Conduit

A 2 cm-diameter Tissue Orb prototype was assembled sterilely with a microporous polycaprolactone central conduit. Human bone marrow-derived mesenchymal stem cells (MSCs) were cultured at 400,000 cells/ml in Organoid Media composed of 1:1 Hepatocyte Culture Medium™ (Lonza Biosciences) and Endothelial Cell Growth Medium-2(TM) (Lonza Biosciences) supplemented with 20 ng/ml hepatocyte growth factor, 10 ng/ml oncostatin M, and 2.5 M dexamethasone. The orb assembly was placed within the RPM to simulate microgravity and cultured in a 37° C. and 5% CO₂ incubator for 2 days. The central conduit was retrieved from the orb after the culture period and analyzed by fluorescence microscopy. Staining by phalloidin for cytoskeleton and DAPI for nuclei showed that MSCs attached and coated the central conduit within the Tissue Orb (Figure F). This example demonstrates that MSCs are able to coalesce upon the Tissue Orb central conduit under microgravity conditions and serve as stromal cells and/or pericytes for other cell types to build upon.

### Example 3. Function and Vascularization of Microgravity Self-Assembled Liver Organoids

Human induced pluripotent stem cell (iPSC)-derived hepatic progenitors were cultured at a density of 500,000 cells/ml in Organoid Media in under normal gravity and under sumulated microgravity applied using an RPM. Conventional iPSC-derived liver organoids were also generated on top of Matrigel or iPSC-derived liver monolayers. After three weeks of culture, the organoids were assayed for expression of hepatic genes, albumin production, and vascular structure.

Organoids produced under microgravity had upregulated by >150X hepatocyte-enriched functional and cell-type-specific genes as determined by next generation mRNA sequencing (RNA-seq).

Over 3 weeks of culture, microgravity self-assembled liver organoids produced sustained and significantly higher levels of albumin per cell than compared to conventional Matrigel organoids. Albumin production is an important measure of hepatocyte function. The levels of albumin produce by microgravity self-assembled liver organoids at days 14 and 22 were comparable to levels produced by human hepatocytes in vivo, as depicted in FIG. 4B.

Co-culture of iPSC-derived hepatic progenitors, human umbilical vein endothelial cells (HUVECs), and MSCs at a ratio of 10:10:1 in Organoid Media under simulated microgravity led to formation of multi-lineage liver organoids with internal endothelial cell organization resembling vascular networks, as assessed by confocal microscopy and staining for CD31 and HNF4α. These examples demonstrate that human stem cell-derived liver organoids self-assembled in microgravity and were more functional than conventional Matrigel-dependent organoids while exhibiting internal microvascular organization.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A tissue culture system, comprising
a spherical tissue culture vessel, comprising:
an inner volume having substantially spherical geometry lacking corners, edges, flat planes, and protrusions; and
two or more ports accessing the inner volume, wherein the two or more ports comprises a first port and a second port, and wherein the first port and second port are configured to receive the opposing ends of a conduit.

2. The tissue culture system of claim 1, wherein
the spherical inner volume of the spherical tissue culture vessel is formed by joining complementary bodies comprising hemispherical volumes.

3. The tissue culture system of claim 2, wherein
each of the two complementary bodies forming the spherical inner volume of the tissue culture vessel comprises a block of material having a hemispherical depression therein.

4. The tissue culture system of claim 2, wherein
each of the two complementary bodies forming the spherical inner volume of the tissue culture vessel comprises a hemispherical demiorb.

5. The tissue culture system of claim 2, wherein
each of the two complementary bodies forming the spherical inner volume of the tissue culture vessel is mounted in a support block.

6. The tissue culture system of claim 1, wherein
the tissue culture vessel comprises a transparent material.

7. The tissue culture system of claim 1, wherein
the tissue culture vessel comprises one or more ancillary ports accessing the inner volume thereof.

8. The tissue culture system of claim 7, wherein
the one or more ancillary ports accessing the inner volume of the culture vessel comprise at least one input port and one output port in connection with elements for flowing material into and out of the inner volume of the tissue culture vessel.

9. The tissue culture system of claim 1, further comprising
a conduit traversing the inner volume of the vessel;
wherein the conduit comprises: a blood vessel or a tubular body comprising a biocompatible material;
wherein the conduit further comprises an inner lumen; and
wherein the ends of the conduit are in connection with a source of liquid medium and elements for flowing culture medium through the conduit.

10. The tissue culture system of claim 9, wherein
the conduit comprises a medical textile.

11. The tissue culture vessel of claim 9, wherein
the conduit is functionalized with bioactive molecules.

12. The tissue culture vessel of claim 11, wherein
the bioactive molecules comprise angiogenic agents.

13. The tissue culture vessel of claim 1, wherein
the central conduit comprises a cellular scaffold.

13. The tissue culture system of claim 1, further comprising
mechanical components configured to rotate such that the culture vessel inner volume experiences a state of reduced gravity or weightlessness.

14. The tissue culture system of claim 13, wherein
the mechanical components comprise a random positioning machine, clinostat, or magnetic levitation device.

15. A method of producing a vascularized cell culture product, comprising
introducing progenitor cells suitable for the formation of a selected cell culture product to a culture vessel of any of claims 1-14, wherein the culture vessel is filled with a first liquid medium;
flowing a second liquid medium through the central conduit; and
performing the sequential introduction of selected biologically active molecules into the first liquid medium of the spherical volume of the culture vessel and/or the second liquid medium flowed through central conduit;
wherein the sequential introduction of selected biologically active molecules is performed in a protocol suitable for the formation of the selected cell culture product; and
wherein a cell culture product comprising a macroscopic vascularized multicellular body is produced.

16. The method of claim 15, wherein
the tissue culture product is an article selected from the group consisting of an organoid, a tissue, or an organ.

17. The method of claim 16, wherein
the selected organoid, tissue, or organ type is selected from the group consisting of liver, kidney, pancreatic, pancreatic, muscle, prostate, lung, cardiac, thyroid, intestinal, mammary, prostate, muscle, brain and dermal tissue.

18. The method of claim 16, wherein
the selected tissue type is liver.

19. The method of claim 15, wherein
the cells within the tissue culture vessel are subjected to a state of reduced gravity or weightlessness during a portion or the entirety of the culture process.

20. The method of claim 19, wherein
the culture vessel is rotated by mechanical components to create a state of reduced gravity or weightlessness.

21. The tissue culture system of claim 20, wherein
the mechanical components comprise a random positioning machine, clinostat, or magnetic levitation device.

22. The method of claim 10, wherein
the state of reduced gravity or weightlessness is achieved by performing a portion or all of the culture process in space.

23. A method of producing a vascularized cell culture product, comprising
introducing progenitor cells suitable for the formation of a selected cell culture product to a culture vessel containing a liquid medium;
performing the sequential introduction of selected biologically active molecules into the liquid medium of the culture vessel;
wherein the sequential introduction of selected biologically active molecules is performed in a protocol suitable for the formation of the selected cell culture product;
wherein a cell culture product comprising a macroscopic vascularized multicellular body is produced; and
wherein the cells within the tissue culture vessel are subjected to a state of reduced gravity or weightlessness during a portion or the entirety of the culture process.

24. The method of claim 23, wherein
the culture vessel is rotated by mechanical components to create a state of reduced gravity or weightlessness.

24. The tissue culture system of claim 23, wherein
the mechanical components comprise a random positioning machine, clinostat, or magnetic levitation device.

25. The method of claim 24, wherein
the state of reduced gravity or weightlessness is achieved by performing a portion or all of the culture process in space.

26. The method of claim 14, wherein
the culture vessel further comprises a central conduit through which a liquid medium is flowed.
